(19) United States
(12) Patent Application Publication
(10) Pub. No.: US 2015/0088542 A1
(43) Pub. Date: Mar. 26, 2015

(54) SYSTEM AND METHOD FOR CORRELATING EMOTIONAL OR MENTAL STATES WITH QUANTITATIVE DATA

(71) Applicant: Be Labs, LLC, Seattle, WA (US)

(72) Inventor: Edward Balassanian, Seattle, WA (US)

(21) Appl. No.: 14/498,994

(22) Filed: Sep. 26, 2014

Related U.S. Application Data
(60) Provisional application No. 61/882,971, filed on Sep. 26, 2013.

(51) Int. Cl. G06F 19/00; G06Q 30/06; A61B 5/16

(52) U.S. Cl. CPC G06F 19/363; A61B 5/165; G06Q 30/0631; G06F 19/3487
USPC 705/2

(57) ABSTRACT

Techniques are disclosed relating to computer facilitated determination of a correlation between an individual's emotional or mental state and influential data corresponding to the individual. The correlation may be determined over a particular time interval or substantially in real-time. Influential data may be provided from one or more data sources (e.g., a device) associated with the individual. The report may be communicated in various forms including graphs and other visual representations. The report may be communicated to the user via various communication channels such as emails, text messages, webpages, and other digital or physical form.

This application claims the benefit of U.S. Provisional Application No. 61/882,971, filed on Sep. 26, 2013, which is incorporated by reference herein in its entirety.

### BACKGROUND

1. Technical Field

This disclosure relates to techniques for correlating an individual's emotional or mental state (e.g., mood, stress, feelings, etc.) with data including quantitative data.

2. Description of the Related Art

An individual's emotional or mental state such as the individual's mood or stress may be determined based on a variety of tests or assessment tools. For example, a few well-known tools directed to evaluating an individual's emotional or mental state include the Profile of Moods States (POMS), the Interpersonal Reactivity Index (IRI) and the Maslach Burnout Inventory (MBI). Each of these tests or tools contains a series of tailored questions used to determine a specific emotional or mental state of the individual answering the questions. These tests are often administered, graded and interpreted by a professional.

### SUMMARY

Techniques are disclosed relating to determining a correlation between a user's emotional or mental state and influential data for the user. In an embodiment, a computer system may include a processor and one or more memories that store executable instructions. The instructions may be executable by the processor to receive mental health data associated with a mental state of a user and/or mental health of the user. The mental health data may be inputted by a networked device such as a health or fitness device configured to communicate with the computer system via a network.

In addition to receiving the input from the user, the instructions may be executed to compile or aggregate influential data corresponding to the user. The influential data may include quantitative data. The instructions may be executed to determine a correlation between the mental health data and the influential data. The correlation may be determined or tracked over a time interval.

The instructions may be executed to generate a report that includes the correlation. The report may be customized to include a variety of contents including contents requested by the user. The report may also include recommendations related to one or more variables of the correlation. The report may be updated when a value of the one or more of the variables changes.

The instructions may be executed to store in a memory the report, the mental health data and/or the influential data. All or any portion of the data stored in the memory may be communicated to the user via a communication channel. The user may specify the way in which the data is to be communicated and the frequency of communication.

In another embodiment, a computer-implemented method may include comparing data associated with one user (e.g., mental health data, influential data, correlation, etc.) with those associated with another user (e.g., mental health data, influential data, correlation, etc.) or other users. Comparing data associated with one user with those associated with other user or users may include, for example, mapping the mental health data indicative of an emotional or mental state status of one user to the mental health data indicative of the emotional or mental state status of one or more other users. The user may view, as an example of a result of the comparing, the user's data, trends, or correlations are similar and/or different to those of other users.

In yet another embodiment, a method may include a user selecting an alert configuration corresponding to a determination of the correlation. The user, may, provide one or more rules or criteria governing the alert configuration. When the computer system detects that the rules or criteria have been met, it may trigger the alert and cause the user to be alerted. Upon being alerted, the user may provide data including mental health data to the computer system for the determination of the correlation.

Various embodiments are disclosed related to a type of computer-implemented service that may facilitate the determination of a correlation between user's mental or emotional state and influential data corresponding to the user.

Various embodiments are disclosed related to a computer-implemented solution for the trending, tracking, monitoring, or reporting of a user's mental or emotional state with regard to influential data corresponding to the user.

Various embodiments are disclosed related to analyzing a correlation between a user's mental or emotional state with regard to influential data corresponding to the user and providing a recommendation for a product or service. The recommendation may be made based on predicted changes to the user's mental or emotional state based on the correlation.

### BRIEF DESCRIPTION OF THE DRAWINGS

FIG. 1 illustrates an embodiment of a computer system configured to determine a correlation between a user's mental health data and influential data according to the disclosure.

FIG. 2 illustrates a flow diagram of an embodiment of a computing system performing various methods according to the disclosure.

FIG. 3 illustrates a flow diagram of another embodiment of a computing system performing various methods according to the disclosure.

FIGS. 4-5. illustrate embodiments of variations and/or components of the computer system illustrated in FIG. 1.

FIG. 6 illustrates a flow diagram of a computer system carrying out a method according to the disclosure.

FIG. 7 illustrates a flow diagram of a computer system carrying out another method according to the disclosure.

FIGS. 8A and 8B illustrate embodiments of various displays or reports according to the disclosure.

FIGS. 9A and 9B illustrate embodiments of various additional displays or reports according to the disclosure.

FIG. 10 illustrates a particular embodiment of a report displayed on an interface of a device executing an application.

FIG. 11 illustrates another particular embodiment of a report displayed on an interface of a device executing an application.

This specification includes references to “one embodiment” or “an embodiment.” The appearances of the phrases “in one embodiment” or “in an embodiment” do not necessarily refer to the same embodiment. Particular features, structures, or characteristics may be combined in any suitable manner consistent with this disclosure.

“Comprising.” This term is open-ended. As used in the appended claims, this term does not foreclose additional structure or steps. Consider a claim that recites: “An apparatus comprising one or more processor units . . . . ” Such a claim does not foreclose the apparatus from including additional components (e.g., a network interface unit, graphics circuitry, etc.).

“First,” “Second,” etc. As used herein, these terms are used as labels for nouns that they precede, and do not imply any type of ordering (e.g., spatial, temporal, logical, etc.). For example, in a processor having eight processing elements or cores, the terms “first” and “second” processing elements can be used to refer to any two of the eight processing elements. In other words, the “first” and “second” processing elements are not limited to logical processing elements 0 and 1.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

As used herein, the term “coupled to” may indicate one or more connections between elements, and a coupling may include intervening elements.

Various units, circuits, or other components may be described or claimed as “configured to” perform a task or tasks. In such contexts, “configured to” is used to connote structure by indicating that the units/circuits/components include structure (e.g., circuitry) that performs the task or tasks during operation. As such, the unit/circuit/component can be said to be configured to perform the task even when the specified unit/circuit/component is not currently operational (e.g., is not on). The units/circuits/components used with the “configured to” language include hardware—for example, circuits, memory storing program instructions executable to implement the operation, etc. Reciting that a unit/circuit/component is “configured to” perform one or more tasks is expressly intended not to invoke 35 U.S.C. §112(f) for that unit/circuit/component.

### DETAILED DESCRIPTION

Web-based tools may implement emotional or mental state tests or assessment tools in determining a user's (e.g., an individual's) emotional state. While the user may take such a test over the web without the assistance of a professional, the user may receive no more than a generic explanation of the test result which might not correlate potential reasons as to why the user has been evaluated for a specific emotional or mental state.

Frequency of testing may be an additional issue in the testing and/or assessment of a user's emotional or mental state. A user's emotional or mental state may change from time to time and may vary based on various events in the user's life. In order to track these changes, a baseline of the user's emotional or mental state may be established with which subsequent emotional or mental state may be compared. When a user takes an emotional or mental state test frequently, the user may gather a larger quantity of emotional or mental state results and associated data. Because emotional or mental state tests or assessment tools generally include a number of questions to which a user must answer, it can be a time consuming activity especially if the user takes the tests frequently.

Influential factors and the data associated therewith, such as ongoing events in a user's life may impact the user's emotional or mental state. For example, weather condition may impact the user's emotional or mental state. Correlating outside influential factors with the emotional or mental state of the user may provide additional insights into the emotional or mental state of the user and variations in the states.

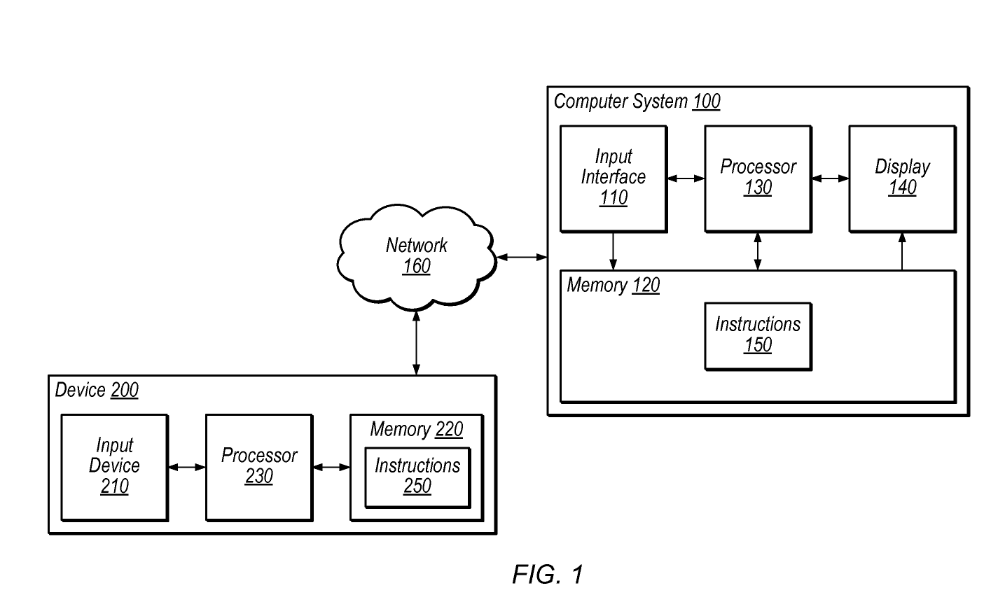

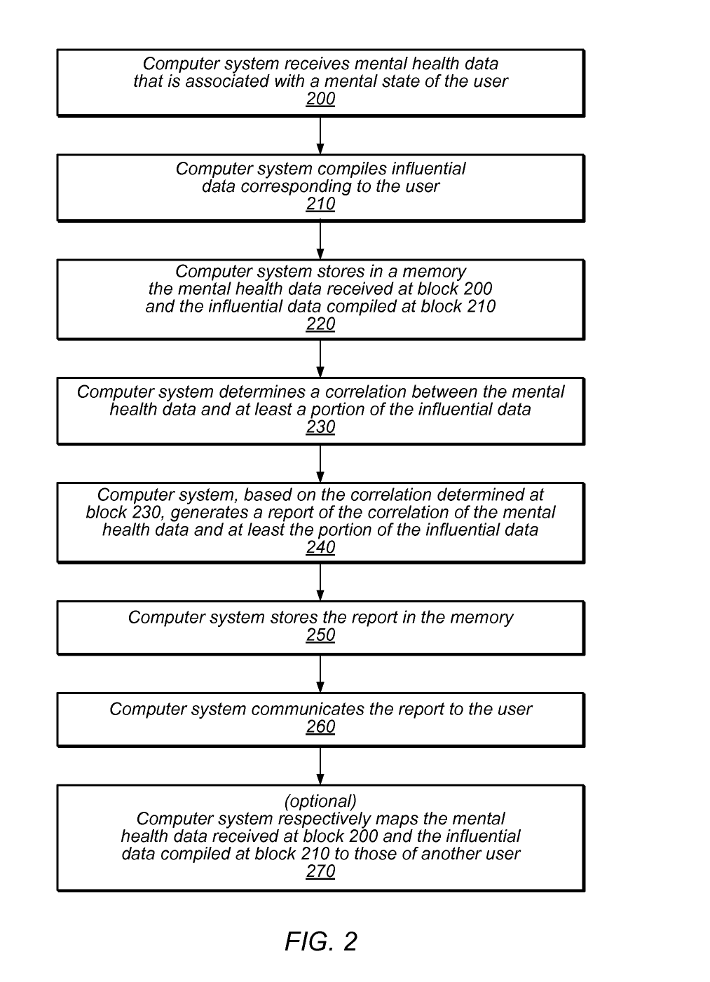

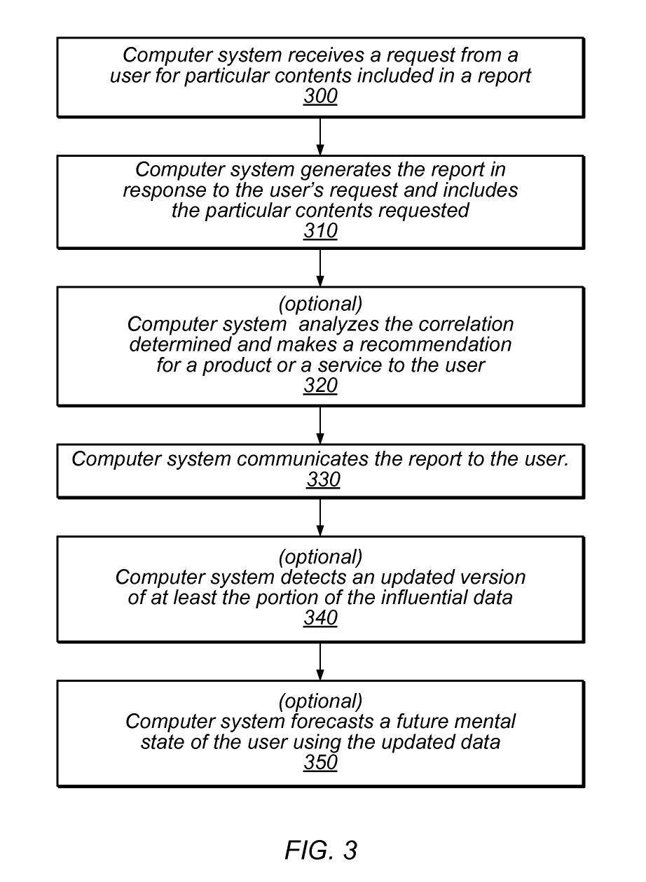

FIG. 1 illustrates a computer system that may be configured to carry out various embodiments of a method to correlate mental health data and influential data including those illustrated in FIGS. 2-3. In particular, FIG. 1 illustrates computer system 100, which may also be referred to as a computer device or a computing device. Computer system 100 may include input interface 110, memory 120, and a processor 130, and a display 140. Computer system 100 may be configured to communicate via network 160. In the illustrated embodiment, memory 120 stores executable instructions 150. In some embodiments, computer system 100 may be a mobile computing device such as a mobile phone or smartphone.

Computer system 100 may be associated with an operator or administer. In one non-limiting embodiment, computer system 100 may be associated with a mental health monitoring and analysis service. In another embodiment, however, computer system 100 may be associated with an entity separate from the mental health monitoring and analysis service.

A user of computer system 100 may be an individual, a client, a customer, or a subscriber of a mental health monitoring and analysis service. In a non-limiting embodiment, a user may have an account with the mental health monitoring and analysis service. The user's account may be accessible by the user and others who have been authorized to access the user's account. The user may access the account in a variety of ways, including in person or via network 160.

Memory 120, in the illustrated embodiment, stores instructions 150 for execution by processor 130, and may also store various types of data generated or used during operation of customer device 100. For example, memory 120 may store data received by computer system 100 such as mental health data associated with a mental state of a user. Any suitable memory technology may be employed for memory 120. Moreover, memory 120 is one example of a non-transitory computer-readable or computer-accessible medium capable of storing instructions for execution by a processor, such as processor 130. It is noted that other examples of non-transitory computer-readable media for storing such instructions are possible, such as various forms of RAM, ROM, readable and/or rewritable optical disc media, magnetic disc media, nonvolatile memory, and the like.

Processor 130, in the illustrated embodiment, is coupled to memory 120 and configured to execute instructions 150. Processor 130 need not be limited to any particular type of device architecture, and multiple instances of processor 130 (or multiple processing cores within processor 130) may be employed.

Network 160, in various embodiments, may be any suitable network technology for facilitating communication between computer system 100 and another computer system, a computing device, or a server. For example, network 160 may include wired or wireless network or telecommunications technology, or a combination of these.

Computer system 100 may, through input interface 110, receive input. Interface 110 may include a keyboard, a touch screen, a mouse, and/or other types of interface that may facilitate computer system 100 in receiving input from a user. In some embodiments, input interface 110 may operate in conjunction with software implemented by instructions 150 that are executed by processor 130. In other embodiments, however, computer system 100 may receive input from a user without going through input interface 110. For example, computer system 100 may receive input data by downloading the data via network 160.

Computer system 100 may receive user input from device 200 that is configured to communicate with computer system 100 via network 160. Device 200, when connected to network 160 may be referred to as a networked device. Device 200 may also be referred to as a health device or a user input device. In some embodiments, device 200 may include at least one of the following: exercise equipment, an activity monitoring device, a medical device, a scale, a body fat measurement device, a blood sugar measurement device, or a blood pressure measurement device. In certain embodiments, device 200 may include a health device that is selected from the group consisting of: exercise equipment; activity monitoring device; scale; body fat measurement device; blood sugar measurement device; and blood pressure measurement device. In other embodiments, however, device 200 may include a mobile computing device such as a mobile phone or smartphone. Device 200 in turn may include input device 210, memory 220, processor 230, and may be configured to communicate via network 160. In the illustrated embodiment, memory 220 stores executable instructions 250. Device 200 may additionally include a display (not illustrated).

Input device 210 may include any device through which a user may input data. For example, input device 210 may include a keyboard, a touch screen, a mouse or other types of interface that may facilitate data being inputted. Input device 210 may also include sensors or other detectors that may detect or measure data associated with the user. For example, input device 210 may include sensors for blood sugar level, heart rate, pulse, weight, body fat content, blood pressure level, body temperature, activity, duration of rest or activity, and other data associated with a user. In some embodiments, input device 210 may operate in conjunction with software implemented by instructions 250 that are executed by processor 230.

FIG. 2 illustrates an embodiment of a method correlating a user's mental health data (e.g., indicative of a mental or emotional state) based on quantitative data using a computer system such as computer system 100.

Operation begins at block 200 where a computer system receives mental health data from a user. The computer system may receive mental health data that is associated with a mental or emotional state of the user.

The mental health data may be received by the computer system based on an alert configuration entered by the user. The user may enter into the computer system an alert configuration that sets, for example, one or more rules or conditions for the alert configuration to be triggered for the user to provide the mental health data. The computer system may then determine if the rules or conditions have been met to trigger the alert. When then computer system determines that the rules or conditions for the alert configuration have been met, the computer system may therefore alert the user for the user to input the user's mental health data. The computer system in turn receives the mental health data from the user as a response to the alert transmitted by the computer system.

As used herein, a “mental state” may include mean emotional state, feeling, perception, state of mind, state of well-being, mood, emotion, sensation, awareness, belief, recognition, sentiment, cognition, or any other psychological notions. A “mental state” may include, without limitation, happiness, joy, anger, empathy, shyness, proudness, sadness, fearfulness, fearlessness, worry, surprise, indifference, excitement, disappointment, humiliation, hopefulness, compassion, devotion, motivation, optimism, anxiety, relaxation, depression, mournfulness, elation, moodiness, or other types of mental or emotional state.

The mental state of the user may be an existing or present mental state. The mental state of the user may also be a past mental state. In one embodiment, the mental health data may include a relative indication of mental and/or emotional state which may include mental health and/or emotional health. For example, mental health data may include a score, a rating, a risk factor, an estimate, a calculated result, or any other types of measurements or representations of a user's mental or emotional state.

Operation proceeds to block 210 where the computer system compiles or aggregates influential data corresponding to the user. The computer system may receive the influential data from device 200. The influential data includes quantitative data.

As used herein, “influential data” include any information, factor, event, condition, experience, or occurrence that may directly or indirectly influence, affect, be reacted upon, or otherwise have an impression on a user. “Influential data” may include, without limitation, data about life events, natural disasters, financial events, physical location, location, stock market status, email volume, number of appointments on a schedule, frequency of appointments on a schedule, types of appointments on a schedule, schedule status, deadline status, goal status, exercise status, exercise frequency, exercise type, diet, consumption, weight, sports team statistics, polls, body fat percentage, financial events, time, day, month, physical health, weather, or any such information that may either directly or indirectly impact, affect, influence, or otherwise have an impression on the user.

Influential data may include quantitative data that can be counted or expressed numerically. For example, quantitative data may be the Dow Jones Industrial Average on a given day, the number of upcoming appointments on a schedule, a football game score for a particular game, a calorie intake for a meal, etc.

Operation proceeds to block 220 where the computer system stores in a memory the mental health data received at block 200 and the influential data compiled or aggregated at block 210. The memory may be similar to memory 120 illustrated in FIG. 1.

Operation proceeds to block 230 where the computer system determines a correlation between the mental health data and at least a portion of the influential data. That is, the computer system may determine a correlation between the mental health data and one or more data items of the influential data. For example, the computer system may determine that there is a correlation between the mental health data indicative of a happy mental state and a number or an increase of the Dow Jones Industrial Average. As another non-limiting example, the computer system may determine that there is a correlation between the mental health data indicative of a sad mental state and an unfavorable football game final score.

As used herein, “correlation” means a relation, a dependence, a correspondence, or a connection between at least two parameters, factors, or variables. A “correlation” may include, without limitation, a mathematical, statistical, or other types of measures of how the at least two variables may change in relation to one another. Furthermore, a “correlation,” as used herein, may include a positive correlation or a negative correlation.

In one particular embodiment, the computer system may determine a correlation between the mental health data and at least a portion of the influential data over a period of time. For example the computer system may track the changes or variations of the mental health data over the period of time. The computer system may, for example, track how the mental health data moves in correlation with movements of the Dow Jones Industrial Average over a month. The computer system may, in another example, provide a trend of the correlation between the user's mental health data and football game scores over the entire football season.

Operation proceeds to block 240 where the computer system, based on the correlation determined at block 230, generates a report of the correlation of the mental health data and at least the portion of the influential data. For example, the report may include an analysis or an explanation of the correlation. The report may be in the form of a compilation of statements, a graph, a chart, a calendar, a diary, a summary, a list, a table, a diagram, a histogram, other forms of representation and/or any combination of these forms. The report may be in a digital form, an electronic form, or physical form.

As an alternative or addition to the report, the computer system may generate a recommendation for the user based on the correlation determined at block 230. The recommendation may include suggestions to improve the user's mental health data or mental state. For example, when the computer system determines that there may be a correlation between the mental health data indicative of a happy mental state and the event that the user completed a 30-minute exercise, the computer system may recommend that the user continue with the 30-minute exercise to maintain the happy mental state. In another non-limiting embodiment, when the computer system determines that there may be a correlation between the mental health data indicative of a depressed mental state and the hours of sleep that the user is getting, the computer system may recommend that the user change the hours of sleep in order to change the depressed mental state.

As an alternative or addition to the report, the computer system may forecast a future mental state for the user based on the correlation determined at block 230. In addition to the correlation determined at block 230, the computer system may also use an updated version of at least the portion of influential data for the forecasted mental state. For example, the computer system may determine that there may be a correlation between the mental health data indicative of a happy mental state and the weather condition being sunny. When the weather condition is updated for next day's forecast, the computer system may predict a mental state for the user for the next day based on the correlation determined at block 230 and the weather forecast. That is, when the weather forecast indicates that the weather will be sunny the next day, the computer system may, based on the correlation determined at block 230, predict that the user may be in a happy mental state the next day.

The report may be updated or modified in various embodiments. For example, the computer system may determine that there may be a correlation between the mental health data indicative of a happy mental state and a final score of an inning of a baseball game. When the baseball game proceeds to a subsequent inning, a new final score may replace the earlier final score, and the computer system may accordingly update the report generated at block 240 to reflect the new or updated score which may affect the mental health data of the user.

Operation proceeds to block 250 where the computer system stores the report in the memory. In one non-limiting embodiment, the computer system may store the report in association with the mental health data and influential data stored at block 220. The computer system may store the report in a data structure associated with an identity of the user. For example, the computer system may store the report in an account of the user maintained on or accessible by the computer system.

Operation proceeds to block 260 where the computer system communicates the report to the user. The computer system may communicate the report via a display similar to display 140 illustrated in FIG. 1. The computer system may communicate the report via a network such as network 160 illustrated in FIG. 1. The computer system may communicate the report to the user in digital, electronic and/or physical form.

The computer system may communicate the report to the user once or multiple times. The computer system may communicate the report on a daily basis or over other time intervals. In some embodiments, the computer system may have a default frequency for communicating the report. The default frequency may include daily, weekly, monthly or other reporting frequencies. In other embodiments, however, the computer system may communicate the report in response to the user's request. The computer system may communicate the report on a daily basis when the user specifically requests so. For example, the user may customize the default frequency based on the user's preference. The frequency in which the report may be communicated to the user may be partially or fully customizable based on user request or preference.

Operation proceeds to optional block 270 where the computer system respectively maps the mental health data received at block 200 and the influential data compiled or aggregated at block 210 to those of another user. That is, the computer system may map respective mental health data among multiple users; and the computer system may also map respective influential data among multiple users. The operation occurring at block 270 may take place in other steps during the operation of the computer system. For example, the computer system may map data associated with various users before generating a report.

FIG. 3 illustrates an embodiment of a method for generating a report using a computer system. To the extent that FIG. 3 is consistent with FIG. 2, comments about possible embodiments and implementations made with respect to FIG. 2 apply equally to the description of FIG. 3.

Operation begins at block 300 where a request is received from a user for particular contents included in a report (e.g., report generated at block 240) that relates to the user's emotional or mental state. For example, the request may be provided to the computer system via interface 114. The request may also be provided via network connection 160. The request may include a request for the report to include particular influential data. For example, when a computer system (e.g., computer system 100) compiles or aggregates influential data for the user, the influential data may include a plurality of data items such as temperature on a particular day, the Dow Jones Industrial Average, number of miles the user has run over a week and a variety of other data items. Among the influential data, the computer system may determine a correlation between the mental health data indicative of a happy mental state and the temperature being between 65° F. and 72° F.

Operation proceeds to block 310 where the computer system generates the report in response to the user's request in block 310. In the example where the computer system determines a correlation between the mental health data indicative of a happy mental state and the temperature being between 65° F. and 72° F., the report may include the data item related to the temperature being between the particular range of 65° F. and 72° F. in response to the user's request in block 310. The particular contents included in the report, however, are not limited to the one or more data items of the influential data. The report may be partially or fully customizable based on user request or user preference.

Operation optionally proceeds to block 320 where the computer system (e.g., computer system 100) analyzes the correlation determined (e.g., at block 230) and makes a recommendation for a product or a service to the user. For example, the computer system may analyze a correlation between the mental health data indicative of a happy mental state and influential data showing that the user has achieved a body mass index of 23. Based on the correlation between the mental health data indicative of happiness and the particular body mass index, the computer system may recommend a product to facilitate maintaining or controlling the body mass index. For example, a product recommended may include a fitness or wellness product, a personal fitness monitoring/tracking device, a body mass index monitor, an exercise device or equipment, a food product (e.g., a snack, a beverage, or a meal), a gym membership, or a book on health/wellness topics, etc. The computer system may recommend a service to facilitate maintaining or controlling the body mass index. For example, a service recommended may include a personal training service, a customized meal service, a professional service (physician, dietician, nutritionist, etc.), or counseling service.

In some embodiments, the product or service recommended may be indirectly related to the influential data. For example, if the computer system stores user data that indicates that the user is a smoker, the computer system may recommend product or service related to smoke cessation because smoke may affect the user's body mass index. When the computer system determines that there is a correlation between the mental health data indicative of happiness and the user's body mass index, the computer system may recommend smoke cessation related product or services which may facilitate maintaining or controlling the body mass index.

Operation proceeds to block 330 where the computer system communicates the report to the user. The report may include the recommendations made at block 320. The computer system may communicate the report in a variety of ways including via a digital display, by email, text message, file download, telephone call, other communication formats or a combination of any communication formats. The computer system may communicate the report based on a default setting that may be modified or customized based on user input. The frequency in which the computer system provides the report may depend on a preference indicated by the user. Alternatively, the computer system may provide the report in response to a request from the user.

Operation optionally proceeds to block 340 where the computer system detects an updated version of at least the portion of the influential data. For example, the computer system may detect that the user's blood sugar level has changed from one level to a different level.

Operation optionally proceeds to block 350. When the computer system determines that there is a correlation between the mental health data indicative of a particular emotional or mental state (e.g., happy, sad, angry, etc.) and a level or a range of the user's blood sugar level, then computer system may forecast a future mental state of the user using the updated blood sugar level. Operation ends at block 350.

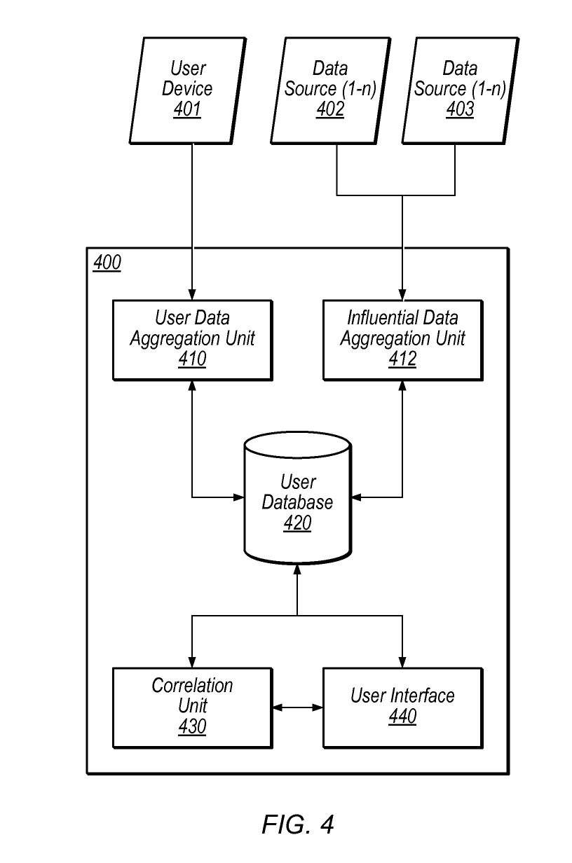

FIG. 4 is an alternative embodiment of a computer system configured to perform the embodiments of the method disclosed herein. Computer system 400 may include similar components as computer 100 such as one or more processors, memory, and instructions. Computer system 400 may include user data aggregation unit 410, influential data aggregation unit 412, user database 420, correlation unit 430, and user interface 440. Computer system 400 may be configured to communicate with user device 401 and one or more data sources 402 and 403. Although computer system 400 is illustrated as having separate components or units, any of the units or components may be combined or divided. To the extent that FIG. 4 is consistent with FIG. 2, comments about possible embodiments and implementations made with respect to FIG. 2 apply equally to the description of FIG. 4.

Computer system 400 may reside, be communicatively coupled to, or include a computer server (not separately shown). Computer system 400 may be configured to manage and store respective accounts for a plurality of users. Computer system 400 may be configured to receive, for example, mental health data for a particular user through the respective account of that user. Computer system 400 may be configured to receive other data including influential data corresponding to the plurality of user. User database 420 may be configured to store data. Computer system 400 may be configured to perform analytics on the data associated with the user and recommend a product or service to the user.

A user may access the respective account maintained on the computer system 400 via an internet connection 405. The user may provide data including mental health data and influential data via user interface 440 of computer system 400 (not separately shown). User interface 440 may be configured to provide a display of a report or data for the user. In one embodiment, user interface 440 may provide a web interface for the users to access or manage their respective accounts. The user may also view data stored in the respective account including mental health data over a selected period of time via user interface 440. The user may also select one or more influential data sources (e.g., 402 and 403) from which influential data may be received. User interface 440 may be configured to provide various views of a user's account. For example, user interface 440 may be configured to provide a view of the account settings, account parameters, preferences, alerts, customizable features, or other attributes related to a user's account.

A user may interact with computer system 400 through user interface 440. User interface 440 may be configured to display any variety of trending reports, specific data reports and alerts, either configured by the system or based on health criteria specified by the user. Additionally, the user can view how the user's trends compare to trends of others (e.g., those similar to the user and others). User interface 440 may be configured to provide the user with the ability to select specific data to be included in comparison and trending reports.

User interface 440 may be configured to provide reporting tools that may display information regarding the influential data that relate to a user's mental or emotional state compared to overall data from data sources 402 or 403 over a specified period of time.

A user may provide data to computer system 400 through user device or user data source 401. User device 401 may be configured to communicate with computer system 400 through the account of the user via a network connection. User device 401 may include health, medical or fitness devices such as a scale, a blood pressure monitor, a blood sugar indicator, a diabetic kit, etc.

User data aggregation unit 410 may be configured to receive and manage data for each of the respective accounts of the users. User data aggregation unit 410 may be configured to manage accounts of the users such as authenticating account access, maintaining user profile or preferences, etc.

Influential data aggregation unit 412 may be configured to receive, collect, retrieve, extract or otherwise aggregate influential data from one or more data sources such as data sources 402 or 403. Each of data sources 402 and 403 may include one or more data sources 1-n. Data sources 402 and 403 may include any data source including, without limitation, weather, Dow Jones Industrial Average, financial data, rainfall, location, time, user-specific data such as amount of exercise, calorie consumptions, exercise parameters such as heart rate or miles per minute, email volumes, appointments on a schedule, a scheduled, specific information such as names, phone numbers or email addresses, etc.

In one particular embodiment, data sources 402 and 403 may be a mobile device (e.g., a smartphone) configured to communicate, integrate, or support one or more health, wellness, fitness or medical devices such as user device 401. In this embodiment, data sources 402 or 403 may be configured to integrate with user device 401 to facilitate the receiving, capturing and reporting of user data including user mental health data. As noted earlier, user device 401 may include a diabetic kit, a blood pressure monitor or other health, wellness, fitness, or medical devices or tools.

In one embodiment, influential data aggregation unit 412 may be configured to process influential data which may include formatting and/or normalizing the influential data. Influential data aggregation unit 412 may be configured to filter, select, categorize the influential data or otherwise organize them for further processing or analysis.

Correlation unit 430 may be configured to analyze mental health data and influential data for the respective users. Correlation unit 430 may be configured to correlate at least a portion of the mental health data with one or more data items of the influential data. For example, correlation unit 430 may be configured to execute, without limitation, one or algorithms, formulae, equations, theorems, hypotheses, numerical methods, computational methods, regression analysis or any combination thereof.

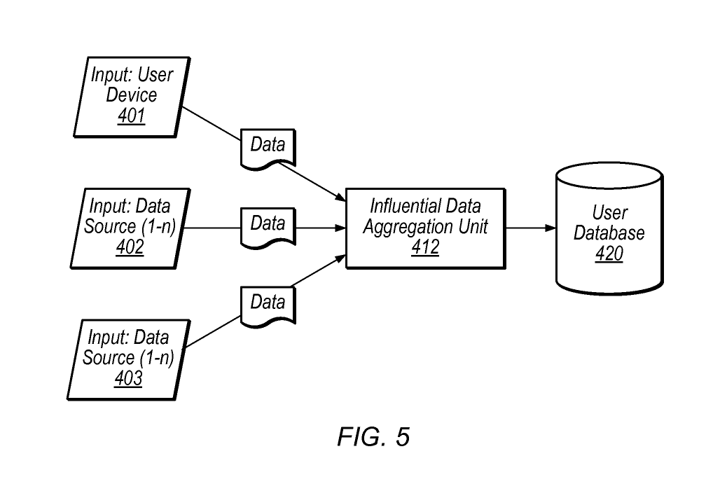

FIG. 5 illustrates an embodiment of user data aggregation unit. The user may register one or more data sources such as those mentioned earlier 401, 402, and 403 with computer system 400. Influential data aggregation unit 412 may be configured to receive data from each of the sources. The data can be received upon request from influential data aggregation unit 412 (e.g., through a data pull) or from the sources on scheduled or ad-hoc basis (e.g., through a data push). In one particular embodiment, the user data aggregation unit may parse, format and store the data associated with a user in user database 420. To the extent that FIG. 5 is consistent with FIG. 4, comments about possible embodiments and implementations made with respect to FIG. 4 apply equally to the description of FIG. 5.

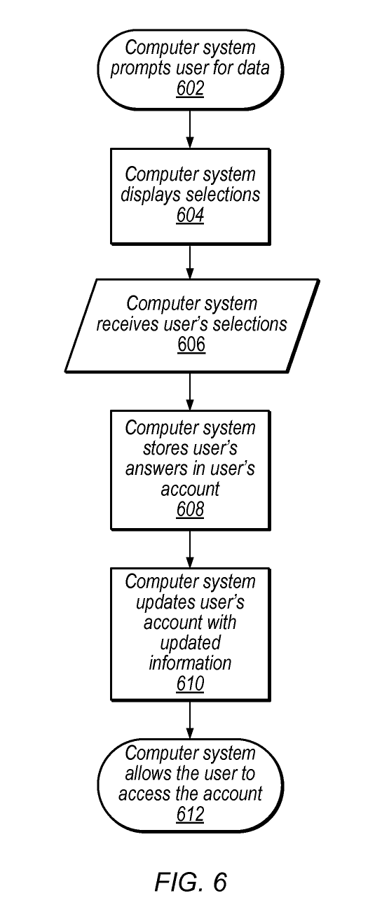

FIG. 6 illustrates an embodiment of a method for inputting mental health data to a computer system. Operation begins at block 602 where a computer system prompts a user at a particular time to input data. For example, the computer system may prompt the user to input mental health data or data indicative of an emotional/mental state at a particular time every day. The user may customize the timing or frequency of the computer system prompting for input.

Operation proceeds to block 604 where the user, upon being prompted, proceeds with inputting mental health data or data indicative of an emotional or mental state via an interface to the computer system. In one particular embodiment, the interface may provide a selection of emotional or mental state or moods either in text or visual representations. The selection may include questions to the user such as, “how are you feeling today?” or “how are you feeling compared to yesterday?” In an alternative embodiment, however, the user may provide inputs without being prompted at block 602.

In yet another embodiment, the selection may be phrase instead of questions. For example, the computer system may display a list of phrases such as “happy,” “sad,” “chill,” “depressed,” “worried” or a variety of other phrases. The user may select one or more phrases that are represent an emotional or mental state or mood of the user.

Operation proceeds to block 606 where the computer system receives answers to questions posed at block 604. In addition, the computer system may receive information including a location of the user, a time at which the responses to the questions were entered, etc.

At block 608, the computer system stores the responses and information received at block 606.

At block 610, the computer system updates an account of the user with the responses and information stored at block 608. The account may be accessible over an internet connection.

At block 612, the user may access the account with the updated information. The user may, for example, access the account, view a report that includes a correlation between mental health data and influential data, view visual representations of the user's mental or emotional data, and view configured alerts or reporting. The user may also request and review trend analysis, recommendation of products or services, and reports that include some or all such contents. Operation ends at block 612.

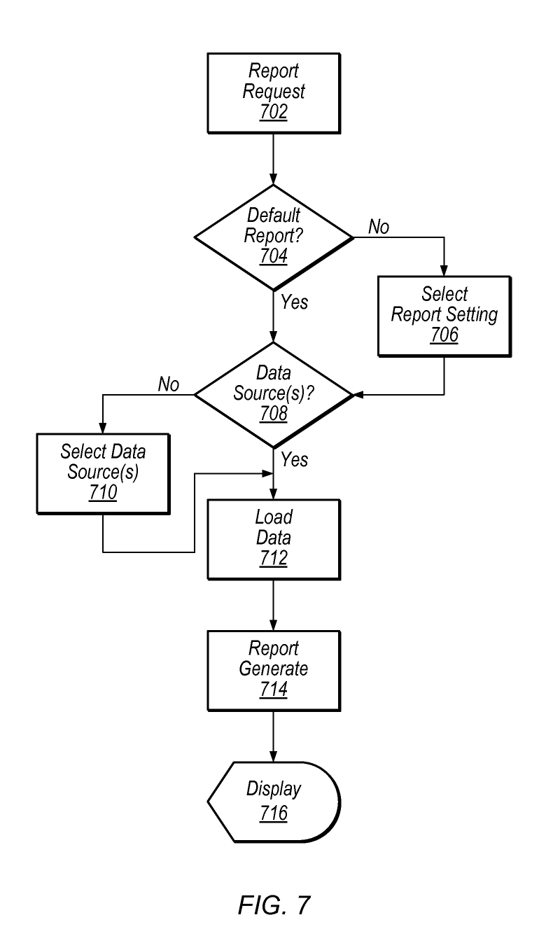

FIG. 7 illustrates an embodiment of a method for determining a correlation between data indicative of a mental or emotional state of a user and influential data.

Operation begins at block 702 where a user requests for the computer system to display a report related to the user's mental or emotional state. The user may request the report via a user input device communicative with the computer system over a network. The user may alternatively request the report through an interface of the computer system.

At block 704, the computer system determines whether there is a default setting or configuration for the report. The default setting may be one that was previously selected by the user. The default setting may be a preferred setting or configuration for the user.

If a default setting is not detected, the operation process to block 706 where the user selects from one or more report setting or configuration. The selected report may include any one of (but not limited to) a chart, graph, visual indicators, numerical representation or any combination of such. The report may be displayed on a webpage, a mobile device, a smart phone or other types of device configured to display images and/or texts.

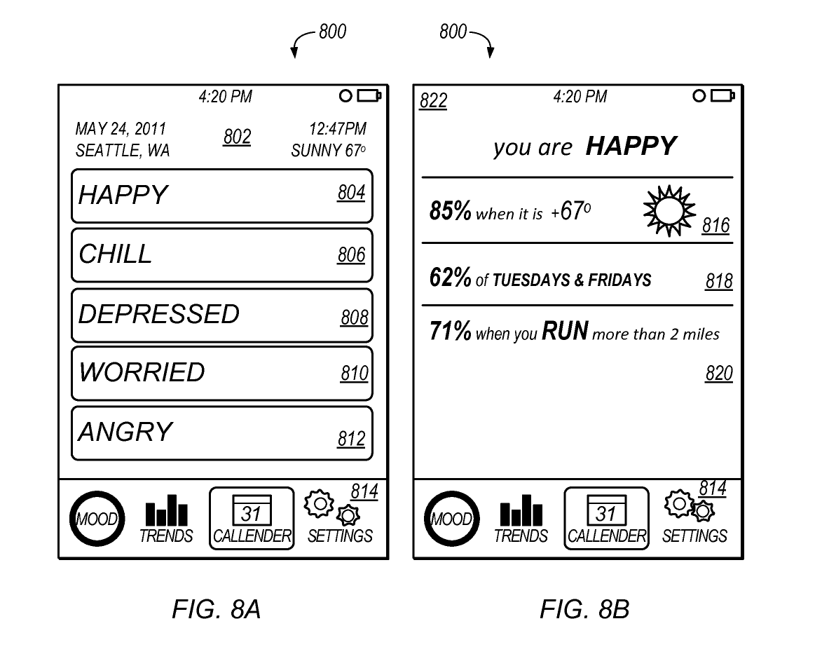

In one non-limiting embodiment, the user may create a user-specific reporting profile that stores default or selected report settings or configurations, data sources or frequency in which the reports are updated. In some embodiments, the user may select a period of time relating to the data analyzed and displayed via the report. Alternatively, the user may set a default period of time for the report. For example, as a default, the user may choose to view trends over the mental health data and/or influential data over a period of days, weeks, months, or years. As discussed in more detail below, the user may specify these and other parameters (e.g., parameters for setting an alarm, alarm configurations, report frequency, report update frequency, etc.) via the function of “SETTINGS” at 814 illustrated in FIGS. 8A-10. In some embodiments, the function of “SETTINGS” at 814 may facilitate user customization of various features of a computer device and/or an application on the computer device.

On the other hand, if the default setting is detected, the operation proceeds to block 708 where the computer system determines whether the user has selected one or more data sources as indicative by, for example, account information for the user's account. The user may adjust or modify the data sources at any given time.

If no data source has been selected, the user may choose particular data sources at block 710.

If previously selected data sources are detected, the computer system proceeds at block 712 to download influential data from at least a portion of the data sources. For example, the computer system may download influential data not limited to those mentioned earlier such as data related to location, time, weather, stock market, email volume, appointment schedule, specific phone numbers, exercise device input, health device input, diet management services, and the like.

At block 714, the computer system generates a report in accordance with the selected setting or configuration at blocks 704-706. The report may include analysis of user data. For example, when a user has inputted data indicating that he is in a “happy” mental or emotional state 20 times in the past 30 days, the computer system may compare that data with the influential data from the one or more data sources that is associated to the user's account. When the influential data include weather and data indicative of the user's activity in the past 30 days, the computer system may compare the occurrences of the user indicating as being “happy” with these influential data. In one particular embodiment, the computer system may calculate that 85% of the time the user indicates as being in a “happy” emotional/mental state, the influential data indicates that the weather is generally sunny and warm. In another example, the computer system may calculate that 71% of the days that the user indicated to be “happy” were days the user ran more than 2 miles.

At block 716, the computer system displays or otherwise communicates the report to the user. In some embodiments, the report may be updated in real-time or substantially real-time when a portion of or all of influential data have been updated. In certain embodiments, the computer system may update a report on an adjustable or customizable time basis. The computer system may analyze the historical mental health data and influential data for the user and make a recommendation for the user. The computer system may recommend a product or service that may facilitate the user in maintaining or controlling the mental health data. Operation ends at block 716.

To the extent that FIGS. 8A-11 are consistent with one another, comments about possible embodiments and implementations made with respect to one of these figures apply equally to the description of other figures.

FIGS. 8A and 8B illustrate non-limiting embodiments of a computer device executing application 800 implementing the various methods disclosed herein. FIG. 8A is one embodiment of an interface on the computer device (e.g., a smartphone) executing application 800. Application 800 may be an application configured to run on any computer device. In one embodiment, application 800 may be an application on a mobile device such as a smartphone. The interface of application 800 may indicate a time at “4:20 PM” and other parameters such as battery life, signal level at location 802. The interface of application 800 may be configured to receive mental health data indicative of a mental or emotion state of a user. For example, the user may input data via a touch screen, a key board or other devices configured to communicate to application 800. The user may be able to select from options such as “Happy” (at 804), “Chill” (at 806), “Depressed” (at 808), “Worried” (at 810), or “Angry” (at 812). Application 800 may be configured to display parameters associated with the user input. For example, application 800 may indicate that the user has selected certain options (“Happy,” etc.) on “May 24, 2011” at “12:47 PM” at a location of “SEATTLE, WA” when the weather is “SUNNY” and temperature is at “67° ” (e.g., 67° F.). These options, as with other examples described herein, are for illustration purposes only and do not limit the disclosure in any way. It is contemplated that any number of options can be provided for the user to select from. It is further contemplated that the selection may be any one of text, color, images, or any combination thereof. Although not illustrated in FIGS. 8A and 8B, colors may be associated with each option. For example, “Happy” may be colored green, whereas “Angry” may be colored red.

Interface of application 800 may at location 814 provide various functions including “MOOD,” “TRENDS,” “CALENDAR,” or “SETTINGS.” The function indicated by “MOOD” may be selectable by the user for application 800 to provide mood, emotional or mental state. The function indicated by “TRENDS” may be selectable by the user for application 800 to provide trends of data or parameters. The function indicated by “CALENDAR” may be selectable by the user for application 800 to provide data for a particular date on the calendar. The function indicated by “SETTINGS” may be selectable by the user for application 800 to configure various settings. For example, by selecting “SETTINGS,” the user may specify a format of a report for a correlation between emotional or mental state and influential data.

FIG. 8B is an illustration of a non-limiting embodiment of a summary of the user's selection of the mental or emotional states using application 800. The display illustrated in FIG. 8B may be displayed when the user chooses a particular function of application 800 (e.g., “MOOD” at location 814). The state summary shown may be directly or indirectly related to the selected options. For example, when the user selected “Happy” as a mental/emotional state, then summary screen 822 may display a report or information about influential data associated with the user selecting the option of being “Happy.” The influential data may be quantitative data. For example, summary screen 820 may be configured to indicate that the user is in a “Happy” mental state 85% of the time when the weather is sunny and the temperature is above 67° F. (at location 816). Summary screen 822 may be configured to indicate that the user is in a “Happy” mental state 62% of the time when it is Tuesdays and Fridays (at location 818). Summary screen 822 may be configured to additionally indicate that the user is in a “Happy” mental state 71% of the time when the influential data indicates that the user has run more than two miles (at location 820).

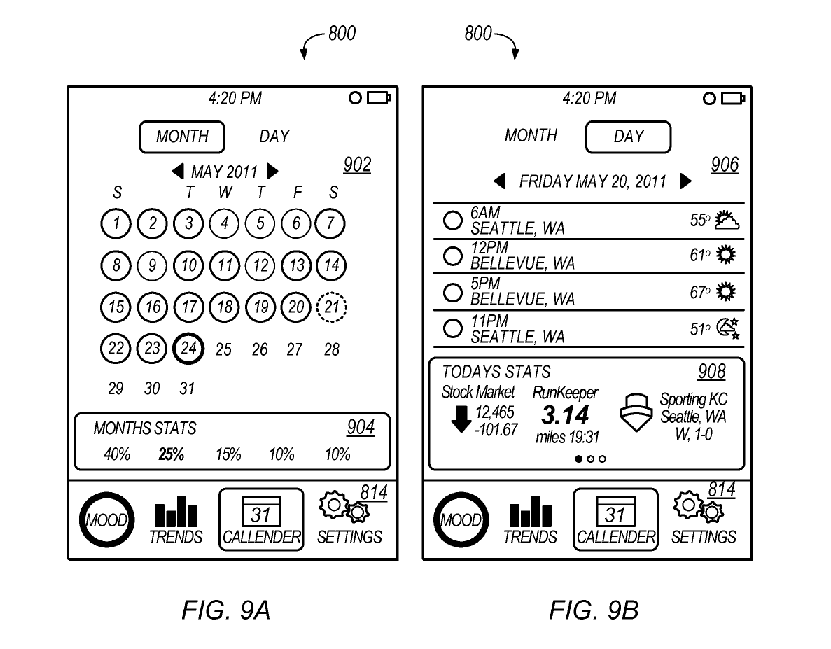

FIGS. 9A and 9B are illustrations of non-limiting embodiments of a report displayed application 800 in accordance with the disclosures herein. FIG. 9A illustrates a calendar interface within application 800. The display illustrated in FIG. 9A may be displayed in response to the user selecting the function of “CALENDAR” at 814. A user may select a specific date, month or other time interval to view a report that includes the user's mental health data and influential data. For example, the user may choose “May 2011” via calendar interface 902. More specifically, the user may choose “May 24, 2011” indicated by the darkened circle around the date “24.” In other embodiments, however, the circles around the days (e.g., numbers) on the calendar may indicate a particular emotional or mental state 25%. For example, the dotted line around the number “21” may be a visual indicator for a particular emotional or mental state of the user on that day.

Calendar interface 902 may display the month of May in 2011 and provide a report including visual indicators for that month. Although not illustrated, the report may be color coded (e.g., red for “Angry”) to facilitate the user's review of days in May of 2011. Calendar interface 902 may provide statistics with regards to the percentage of days a user is in a given mental/emotional state. For example, FIG. 9A illustrates that at location 904, “MONTHS STATS” indicate that the user is at a particular emotional or mental state 25% of the time. In one embodiment, the bolded font of “25%” may correspond to the darkened circle around the number “24” to indicate the particular emotional or mental state which occurred 25% of the time on that day. In other embodiments, however, the bolded font of “25%” and the darkened circle around the number “24” may correspond in other ways or not correspond at all. At location 904, “MONTHS STATS” may additionally indicate that the user has been associated with another emotional or mental state 40% of the time, yet another emotional or mental state 15% of the time, and additional emotional or mental states 10% of the time each.

FIG. 9B illustrates a non-limiting embodiment of daily report 906 that may be viewed by the user on application 800. Daily report 906 may be provided in response to application 800 receiving a command for a particular function from the user. In this example, the user may view a portion or all the times, locations and weather for a particular day such as May 20, 2011 as illustrated. The user may also view data that was received, detected or stored by application 800 for that day. For example, application 800 may provide a report that indicates that at 6 AM on May 20, 2011, the user is located around Seattle, Wash. where the temperature is 55° F. and the weather is partly sunny. The report may indicate that the user is around the same location at 12 PM on that day where the temperature is 61° F. and the weather is sunny. The report may indicate that the user is around the same location at 5 PM on that day where the temperature is 67° F. and the weather is sunny. The report may additionally indicate that the user is around the same location at 11 PM on that day where the temperature is 51° F. and the weather is moon, stars, and some clouds. Report 906 displayed on application 800 may also include, as illustrated at 908, influential data such as a status of the stock market, exercise as recorded by another application (e.g., RunKeeper or other applications), and final score of a game involving a particular sports team. There is no limit to the number of hours, days, weeks, month and years of historical data a user may request, access, or view. Moreover, the report displayed by application 800 is not limited to historical data. Application 800 may display report based on substantially real-time data in some embodiments.

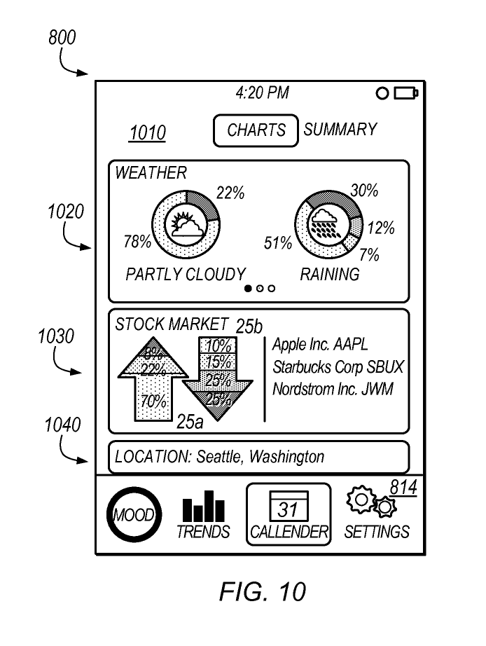

FIG. 10 is an illustration of a non-limiting embodiment of report 1010 that may be displayed on application 800. In this non-limiting example, a user may request, by selecting one of the functions at 814, a chart to access and view how the user's mental/emotional state may relate to at least a portion of the influential data. The influential data may be provided by data sources illustrated in this example such as “Weather” at 1020, “Stock Market” at 1030, and “LOCATION” at 1040. The report may indicate at 1020 that the user has indicated a mental/emotional state as being “Happy” 78% of the time when the weather is partly sunny (or partly cloudy) compared to being “Happy” 51% of the time when there is rain. The report may also indicate at 1020 that the user has indicated a mental/emotional state as being “Sad” 22% of the time when the weather is partly sunny compared to being “Sad” 30% of the time when there is rain. The report at 1020 may additionally indicate that the user has indicated a mental/emotional state as being “Chill” 12% and “Worried” 7% of the time when there is rain.

For example, the report may indicate at 1030 that the user has indicated a mental/emotional state of being “Happy” 70% of the time when the stock market is up (e.g., indicated by the upward arrow 25a) compared to the user indicating being “Angry” 25% of the time when the stock market is down (e.g., indicated by the downward arrow 25b). Other types of mental/emotional state may be indicated by their respective percentages. For example, when the stock market is up (e.g., indicated by the upward arrow 25a), the report may show that the user has indicated another type of mental/emotional state 22% of the time and a third type of mental/emotional state 8% of the time. When the stock market is down (e.g., indicated by the downward arrow 25b), the report indicates that the user has indicated four types of emotional/mental state 25% (e.g., indicating being “Angry”), 25%, 15%, and 10% of the time respectively. The report may also indicate one or more particular stocks (e.g., Apple Inc., Starbucks Corp, and Nordstrom Inc. illustrated at 1030), stock symbols (e.g., AAPL, SUBX, and JWM illustrated at 1030), exchange traded fund, bond or other financial instrument that may be correlated to the user's mental/emotional state.

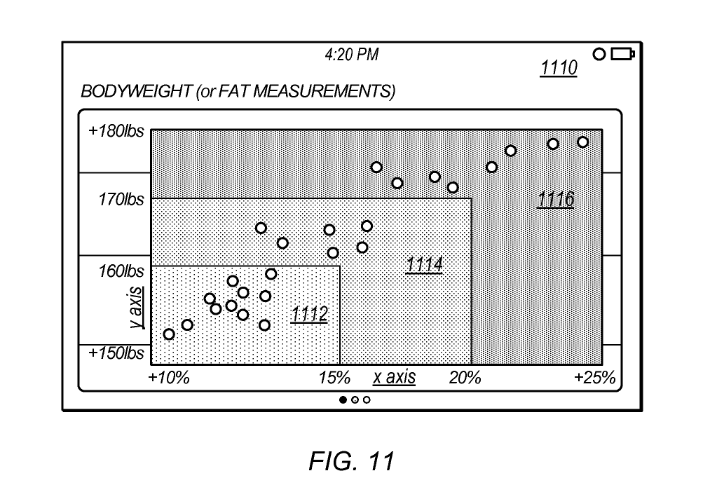

FIG. 11 is an illustration of another non-limiting embodiment of report 1110 that may be generated and/or displayed on application 800. In this example, the report may indicate that the user's mental/emotional state may correlate to influential data such as the user's weight and body fat measurements. Influential data sources such as a wireless scale or wireless body fat measurement device may provide quantitative data for weight, body fat and other wellness related measurements. For example, the user may be associated with a particular emotional/mental state (e.g., at 1112) 10% to 15% (indicated as x axis) of the time when the user's weight is in the range between 150 pounds and just below 160 pounds (indicated as y axis). At location 1114, the user may be associated with a particular emotional/mental state 15% to 20% (indicated as x axis) of the time when the user's weight is in the range between just below 160 pounds and 170 pounds (indicated as y axis). The user may be associated with a particular emotional/mental state (e.g., at 1116) 20% to 25% (indicated as x axis) of the time when the user's weight is in the range between 170 pounds and 180 pounds (indicated as y axis). The x-y plot may facilitate a number of statistical and/or other mathematical analyses or interpretation of the variables associated with the x and y axes.

Although specific embodiments have been described above, these embodiments are not intended to limit the scope of the present disclosure, even where only a single embodiment is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

What is claimed is:

1. A method comprising:
a computer system receiving mental health data from a first user, wherein the mental health data is associated with a first mental state of the first user;
the computer system compiling influential data corresponding to the first user, wherein the influential data includes quantitative data;
the computer system storing the mental health data and the influential data in a memory;
the computer system determining a correlation between the mental health data and at least a portion of influential data;
based on the determining, the computer system generating a report of the correlation of the mental health data and at least the portion of the influential data;
the computer system storing the report in the memory; and
the computer system communicating the report to the first user.

2. The method of claim 1, wherein the plurality of influential data compiled by the computer system includes one or more of: location, stock market status, email volume, appointment schedule, exercise, diet, weight, sports team statistics, body fat percentage, natural disasters, financial events, life events, time, day, month, physical health, or weather.

3. The method of claim 1, wherein the mental health data received by the computer system comprises a relative indication of mental health.

4. The method of claim 1, wherein the computer system is configured to determine the correlation between the mental health data and at least the portion of the plurality of influential data over a period of time.

5. The method of claim 4, further comprising:
the computer system generating a recommendation for the first user based on the correlation between the mental health data and at least the portion of the plurality of influential data over the period of time.

6. The method of claim 1, further comprising:
the computer system receiving an alert configuration from the first user.

7. The method of claim 6, further comprising:
based on the alert configuration, the computer system alerting the first user to input the mental health data.

8. The method of claim 1, further comprising:
the computer system respectively comparing the mental health data of the first user and the influential data of the first user to mental health data of a second user and influential data of the second user.

9. The method of claim 1, further comprising:
based on the correlation between the mental health data and at least the portion of influential data, the computer system forecasting a future mental state of the first user using an updated version of at least the portion of influential data.

10. A method comprising:
receiving, by a computer system, mental health data from a user, wherein the mental health data is associated with a first mental state of the user;
compiling, by the computer system, influential data corresponding to the user, wherein the influential data includes quantitative data;
storing, by the computer system, the mental health data and the influential data in a memory;
determining, by the computer system, a correlation between the mental health data and one or more data items of the influential data;
based on the determining, generating, by the computer system, a report of the correlation of the mental health data and the one or more data items of the influential data;
storing, by the computer system, the report in the memory; and
communicating, by the computer system, the report to the user.

11. The method of claim 10, further comprising:
receiving, by the computer system, a request from the user to include the one or more data items of the influential data in the generated report.

12. The method of claim 10, further comprising:
analyzing, by the computer system, the correlation between the mental health data and the one or more data items of the influential data; and
based on the analyzing, the computer system recommending a product or a service to the user.

13. The method of claim 10, wherein the report is communicated to the user in a form of a graph.

14. The method of claim 10, wherein the report is automatically updated in response to the computer system receiving a new value for the one or more data items of the influential data.

15. A computer system comprising:
a processor; and
a memory having instructions stored thereon that are executable by the processor to cause the computer system to perform operations comprising:
receiving mental health data from a user, wherein the mental health data is associated with a mental state of the user;
aggregating influential data of the user, wherein the influential data includes quantitative data;
storing the mental health data and the influential data in a memory;
determining a correlation between the mental health data and one or more data items of the influential data;
based on the determining, generating a report that includes the correlation of the mental health data and the one or more data items of the influential data;
storing the report in the memory; and
communicating the report to the first user.

16. The computer system of claim 15, wherein operations further comprise:
receiving the influential data of the user from a networked device.

17. The computer system of claim 16, wherein the networked device is a health device.

18. The computer system of claim 17, wherein the health device is selected from the group consisting of: exercise equipment; activity monitoring device; scale; body fat measurement device; blood sugar measurement device; and blood pressure measurement device.

19. The computer system of claim 15 wherein the communicating the report to the user occurs on a daily basis.

20. The computer system of claim 15 wherein the report is in a format of a calendar.
